(19) United States
(12) Patent Application Publication
(10) Pub. No.: US 2013/0023893 A1
(43) Pub. Date: Jan. 24, 2013

(54) ENDOSCOPIC SURGICAL RETRIEVAL APPARATUS AND METHOD WITH SEMI-RIGIDLY EXTENDABLE AND COLLAPSABLE BASKET

(71) Applicant: Rafic Saleh, Aquadilla, PR (US)

(72) Inventor: Rafic Saleh, Aquadilla, PR (US)

(21) Appl. No.: 13/189,041

(22) Filed: Jul. 22, 2011

(51) Int. Cl. A61B 17/94; B23P 11/00

(52) U.S. Cl. 606/113; 29/428

(57) ABSTRACT

An endoscopic surgical device includes a semi-rigid basket comprising a structural combination of resilient, semi-rigid wire frame members, which provide basket shape and form as well as strength, and a web of interspersed flexible threads for enhanced tissue retaining capability.

### TECHNICAL FIELD

The present invention is related to endoscopic surgical devices for capturing and removing tissue pieces and other materials from insides of organs or body cavities accessed endoscopically.

### BACKGROUND

Endoscopic surgery procedures are common in many medical research and practice fields for myriad purposes, for example, to access, diagnose, and treat or excise abnormalities or conditions in interior portions of a patient's body or internal organs that would not be visible or accessible to a surgeon's vision or hands without more extensive or invasive incisions or surgical procedures. An endoscope generally, but not always, comprises some type of rigid or flexible tube (sometimes called a catheter or cannula), a light delivery system to illuminate the target object or tissue, a lens system for transmitting images of the target object or tissue through the tube to the viewer, an eyepiece and/or camera, and often one or more additional lumens to accommodate entry of some kind of medical or surgical instrument or manipulator. Some endoscopic instruments include only the surgical instruments disposed in and deployed by an elongated tube (e.g., catheter or cannula), but not the optical components, in which case they may be used concurrently with another endoscope that does include optical components. Endoscope and endoscopy as used herein are broader terms that encompass various types of such instruments and procedures, including, but not limited to, laparoscopy, bronchoscopy, colonoscopy, and arthroscopy. Endoscopic procedures are common in myriad types of medical specialties and body organs or cavities, including, for example, the gastrointestinal tract, the respiratory tract, the urinary tract, the ear, the reproductive system, normally closed body cavities via small incisions such as the pelvic cavity, joints, organs of the chest, and others. In some of such procedures, various sized and kinds of pieces of tissues are cut or otherwise separated from the organ or target object of the surgery, and, in others, various kinds of other materials are captured and removed from the organs or target objects in the body. In some procedures, such separated pieces of tissue or other materials can be removed by irrigation and suctioning, but others are removed with instruments that are designed to capture and hold them mechanically as they are removed from the organ or body through or with the endoscopic instrument.

An example of an instrument designed to capture and hold separated pieces of tissue or other materials mechanically in endoscopic procedures for removal from organs or bodies is shown in U.S. patent application Ser. No. 11/635,700, filed by Rafic Saleh, on Dec. 6, 2006 (Publication No. 2008/0091215 A1), which is incorporated by reference herein for all that it teaches and discloses. Other examples of such instruments are shown in U.S. Pat. No. 6,656,191, issued on Dec. 2, 2003, to T. Ouchi; U.S. Pat. No. 5,643,283, issued on Jul. 1, 1997, to M Younker; U.S. Pat. No. 5,201,740, issued on Apr. 13, 1993, to N. Nakao; U.S. Pat. No. 5,201,740, issued on Mar. 2, 1993, to N. Nakao; U.S. Pat. No. 6,814,739, issued on Nov. 9, 2004, to D Secrest and M. Younker; and U.S. patent application Ser. No. 11/182,543, filed by N. Nakao on Jul. 15, 2005 (Publication No. 2007/0016224 A1).

The foregoing examples of related art and limitations related therewith are intended to be illustrative and not exclusive, and they do not imply any limitations on the inventions described and claimed herein. Various limitations of the related art will become apparent to those skilled in the art upon a reading and understanding of the specification below and of the accompanying drawings.

### BRIEF DESCRIPTION OF THE DRAWINGS

The accompanying drawings, which are incorporated herein and form a part of the specification, illustrate some, but not the only or exclusive, example embodiments and/or features. It is intended that the embodiments and figures disclosed herein are to be considered illustrative rather than limiting.

In the drawings:

FIG. 1 is a perspective view of an example endoscopic instrument equipped with an example implementation of the surgical retrieval device of this invention;

FIG. 2 is side elevation view of the example endoscopic surgical retrieval device of FIG. 1;

FIG. 3 is a front elevation view of the example endoscopic surgical retrieval device of FIG. 1;

FIG. 4 is an enlarged perspective view of the example endoscopic surgical retrieval device of FIG. 1 looking at the top or open mouth portion of the semi-rigid basket component;

FIG. 5 is a further enlarged perspective view of the example endoscopic surgical retrieval device of FIG. 1 looking at the bottom enclosure portion of the semi-rigid basket component;

FIG. 6 is an enlarged perspective view of the example endoscopic surgical retrieval device of FIG. 1 slightly rotated to look at a side of the semi-rigid basket component; and

FIG. 7 is a perspective view of the example instrument similar to FIG. 1, but showing the semi-rigid basket component partially retracted into the cannula.

### DETAILED DESCRIPTION OF EXAMPLE IMPLEMENTATIONS

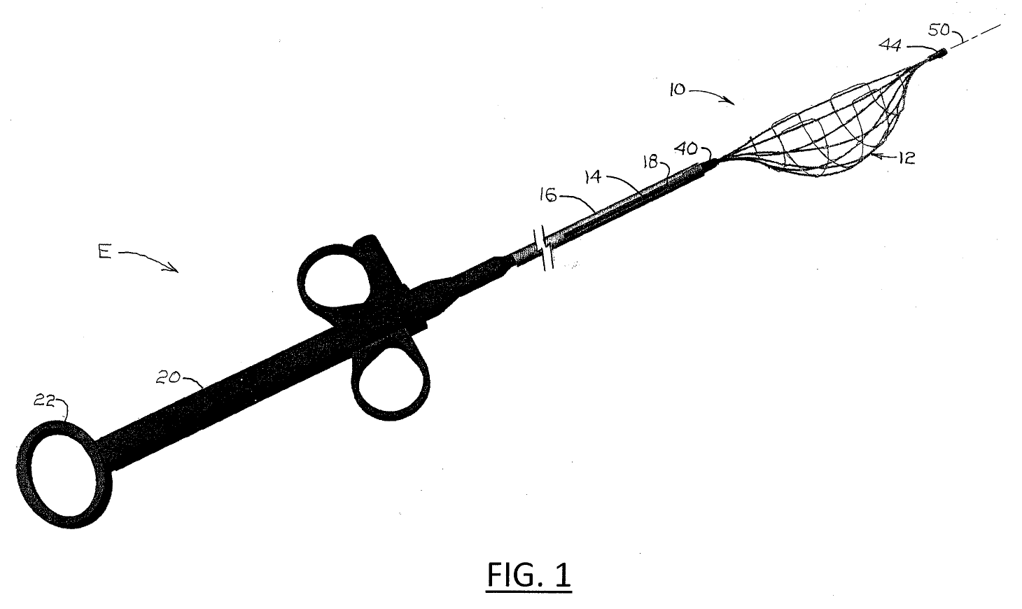

An example endoscopic instrument E equipped with an example implementation of the surgical retrieval device 10 of this invention 10 shown in FIGS. 1-7 can be used for capturing and removing or retrieving tissue pieces and other materials from inside body organs or cavities during endoscopic procedures, including, for example, but not for limitation, gastroenterological, pulmonary, laparoscopic, urological, fluoroscopic, and others. This example endoscopic surgical retrieval device 10 includes a semi-rigid basket 12 comprising an advantageous structural combination of resilient, semi-rigid wire frame members 31, 32, 33, 34, 35, 36, and 37 and flexible thread segments 60 that enhances tissue and material capturing capability, durability, and reliability over prior art baskets with wires and/or mesh-type nets and bags. The resilient, semi-rigid wires in U.S. patent application Ser. No. 11/635,700 provide certain advantages, including durability that withstands the pressures and forces exerted on it during rotations and other manipulations of the device during use in attempting to capture tissue pieces or other materials in endoscopic surgical procedures, which can be significant on such small and somewhat delicate devices. However, they are not reliable for holding captured tissue pieces and other materials that can slip too easily through the spaces between the angularly spaced wires. On the other hand, the soft, sagging, and droopy mesh nets and bags of other prior art devices are too delicate and not very durable and reliable. They often fold, close, and even detach from their mountings due to the pressures and forces resulting from ordinary manipulations of the instruments in body cavities and organs, thus rendering them virtually useless. The structural combination of the wire frame members 31, 32, 33, 34, 35, 36, and 37 with the flexible thread segments 60 in the example basket 12 in FIGS. 1-7 not only provide the durability of the wire frame members 31, 32, 33, 34, 35, 36, and 37, which take the brunt of the pressures and forces applied during manipulations, enhanced by the retaining capability of the flexible thread segments 60, but also the structure enhances the durability and reliability of the flexible thread segments 60 themselves as part of the structure. As will be described in more detail below, the thread segments are laced through and/or attached to a plurality of locations or points along the lengths of the wire frame members 31, 32, 33, 34, 35, 36, and 37, so that, even if one or several segments 60 or parts of the thread 60 should be broken or detached during use, the remaining segments 60 by and large stay in place, which enhances both durability of the device and reliability in retaining the tissue pieces and other materials captured in the basket 12.

The basket 12 of the retrieval device 10 is mounted on the distal end 18 of a wire or tubular tether 14, which extends through a lumen in a catheter or cannula 16, which may or may not have other lumens (not shown) for optical components, irrigation fluids, or other surgical devices or tools (not shown) that may used along with the surgical retrieval device 10. Alternatively, the cannula 16 itself, along with the tether 14 and basket 12, can be deployed through a lumen in another, larger catheter (not shown) that might include lighting and optical components or additional instruments, irrigation lumens, and the like, as would be familiar to persons skilled in the art once they understand this invention. The cannula 16 can be attached to a conventional endoscopic handle 20 for easy manipulation, and the tether 14 can be attached to a telescopic plunger device 22 that extends slideably into the handle 22, where it attaches (not shown) to the tether 14. The plunger device 14 can then be moved longitudinally forwardly and backwardly to extend and retract the distal end 18 of the tether 14 and the semi-rigid basket 12 from and into the cannula 16, as will also be understood by persons skilled in the art once they understand this invention. In this description, the directional prepositions of up, upwardly, down, downwardly, front, back, top, upper, bottom, lower, left, right, and other such terms refer to the device as it is oriented and appears in the drawings and are used for convenience only; they are not intended to be limiting or to imply that the device has to be used or positioned in any particular orientation.

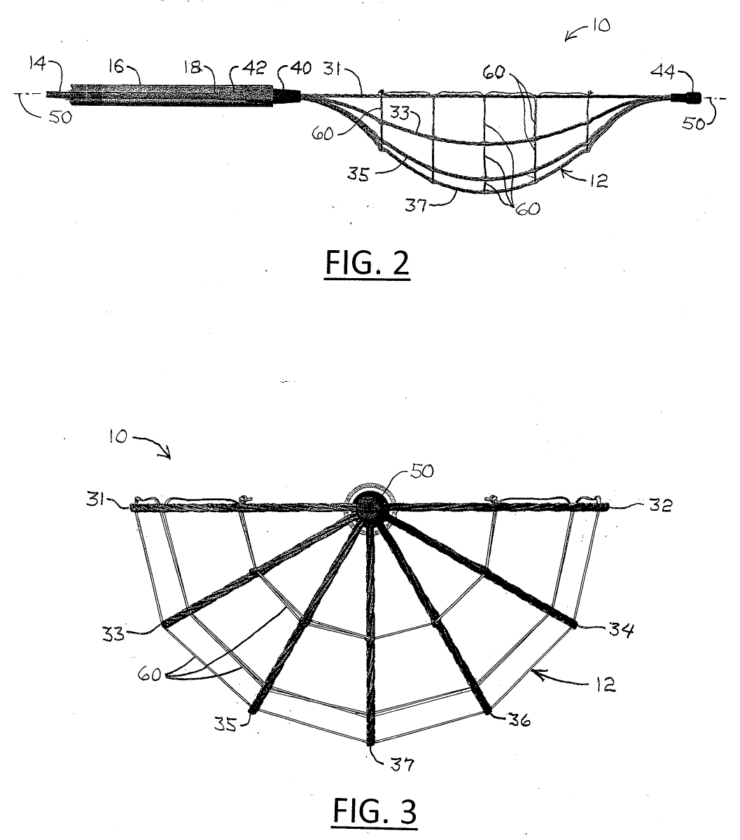

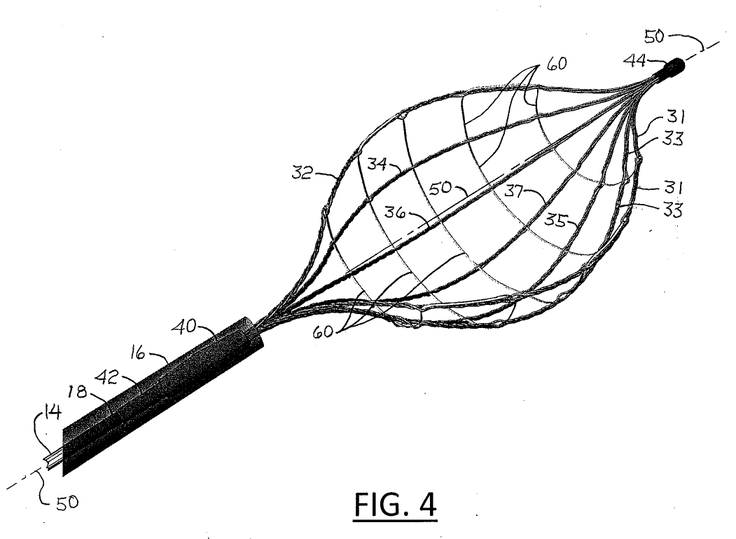

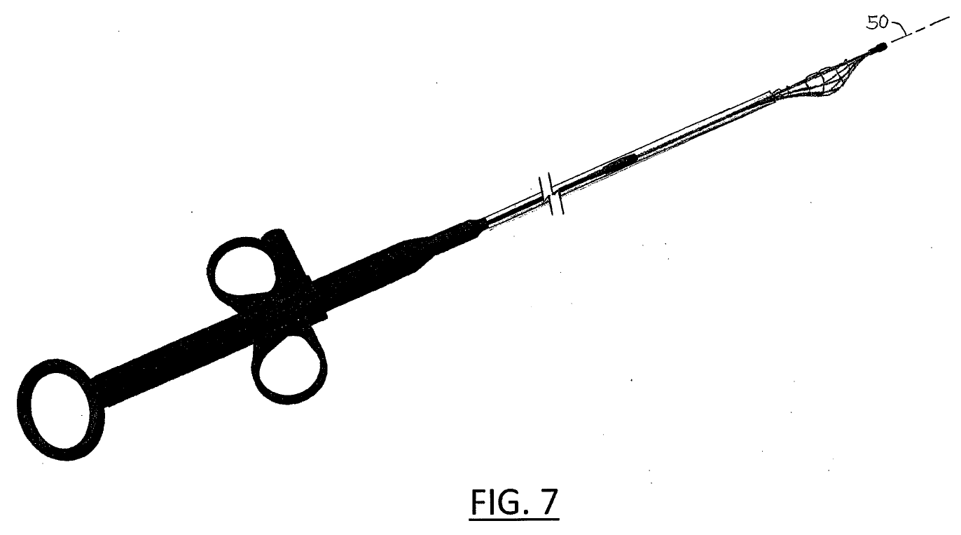

With reference now primarily to FIGS. 2-6 with secondary reference to FIGS. 1 and 7, the semi-rigid basket 12 comprises a plurality of generally longitudinally extending, semi-rigid, resiliently deformable wires, for example, the wires 31, 32, 33, 34, 35, 36, and 37, the proximal ends of which are gathered and fastened together in immovable relation to each other by a ferrule 40 and connected by a coupling 42 to the distal end 18 of the tether 14. The proximal ends of the wires 31, 32, 33, 34, 35, 36, and 37 are gathered and fastened together in immovable relation to each other by another ferrule 44. The wires 31, 32, 33, 34, 35, 36, and 37 are pre-formed into bowed shapes, as best seen in FIGS. 4-6, so that, when they are fastened together in the relative orientations to each other by the proximal ferrule 40 and the distal ferrule 44 as shown in FIGS. 2-6, they flare radially outward from the longitudinal axis 50 in angularly spaced relation to each other about the longitudinal axis 50. The top two wires 31, 32 are shown to be coplanar, thus flaring 180 degrees diametrically opposite each other, in the example device 10 in FIGS. 1-7 to create a wide open mouth for the basket 12 for ease of capturing tissue pieces and other materials (not shown) during endoscopic surgical procedures, while the other wires 33, 34, 35, 36, and 37 are positioned to flare radially outward at more closely spaced angles to each other to for the rest of the structural frame of the basket 12. For example, with the seven wires 31, 32, 33, 34, 35, 36, and 37 of the example device 10 in FIGS. 1-7, the angular spacing between the adjacent wires that form the basket frame can be equal at 30 degrees. Of course, other angular spacings between adjacent wires 31, 32, 33, 34, 35, 36, and 37, including something different than the 180 degrees wide open mouth, can also be used for particular purposes or preferences.

The semi-rigid basket 12 in the example device 10 is completed with flexible thread segments 60 extending transversely between adjacent ones of the wires 31, 32, 33, 34, 35, 36, and 37 at longitudinally spaced distances from each other to effectively make the spaces between the wire frame members 31, 32, 33, 34, 35, 36, and 37 smaller, thus able to capture and retain smaller tissue pieces and other material, which creates a more effective basket than just the wire frame members 31, 32, 33, 34, 35, 36, and 37 by themselves. While not essential, there are advantages to having the flexible thread segments sized in length to become at least marginally taught when the wire frame members 31, 32, 33, 34, 35, 36, and 37 are fully deployed in their flared relation to each other. For example, because of the fastening of the proximal ends of the wire frame members 31, 32, 33, 34, 35, 36, and 37 in immovable relation to each other by the ferrule 40 and fastened by the coupling 42 in immovable relation to the tether 14, rotation of the tether 14 in or along with the cannula 16 by the operator of the device 10 causes the basket 12 to rotate as well. Such rotation of the basket 12 is useful in scooping and other maneuvers to capture tissue pieces and other materials during endoscopic surgery procedures, and the semi-rigid relation of the wire frame members 31, 32, 33, 34, 35, 36, and 37 to each other described above maintains the flared and extended shape of the basket 12 described above and shown in FIGS. 1-6 without folding, drooping, or collapsing during such scooping or other capturing maneuvers, which could be a hindrance to such procedures. By providing the flexible thread segments 60 in lengths that become at least marginally taught when the basket 12 is fully deployed with the wire frame members 31, 32, 33, 34, 35, 36, and 37 fully flared outwardly as described above, the flexible thread segments 60 also retain the shape of the basket 12 without folding, drooping, or collapsing during rotations and other capturing maneuvers of the basket 12, thus do not obscure vision or interfere with the maneuvers while also remaining as effective retaining elements for holding captured tissue pieces and other materials during such procedures.

However, when the basket 12 is retracted back into the cannula 16 (see FIG. 7 wherein the basket 12 is shown partially retracted), the resiliently yieldable, semi-rigid wire frame members 31, 32, 33, 34, 35, 36, and 37 are collapsed by the cannula 16, thereby loosening the flexible thread segments 60, for folding together with the wire frame members 31, 32, 33, 34, 35, 36, and 37 into the cannula 16. In such collapsed condition, the flexible thread segments 60 are pulled by the respective wire frame members 31, 32, 33, 34, 35, 36, and 37 to which they are connected into the cannula 16, which causes the flexible thread segments 60 to form into somewhat folded V-shaped segments between respective adjacent wire frame members 31, 32, 33, 34, 35, 36, and 37 as they are pulled and dragged into the cannula 16. In that formation, the flexible thread segments 60 do not bunch or interfere with the retraction of the collapsed basked 12 into the cannula 16, and they do not interfere with the deployment of the basket 12 out of the cannula 16, either. As the basket 12 is deployed by pushing it out of the distal end of the cannula 16, the resilient, collapsed wire frame members 31, 32, 33, 34, 35, 36, and 37 resume their bowed shapes from the shape memory characteristic of the molecular or crystalline material of which the wire frame members 31, 32, 33, 34, 35, 36, and 37 are made. Suitable materials for the resilient, semi-rigid wire frame members 31, 32, 33, 34, 35, 36, and 37 include, but are not necessarily limited to, stainless steel, nitonal, titanium, or any of a number of synthetic materials that have those properties or characteristics.

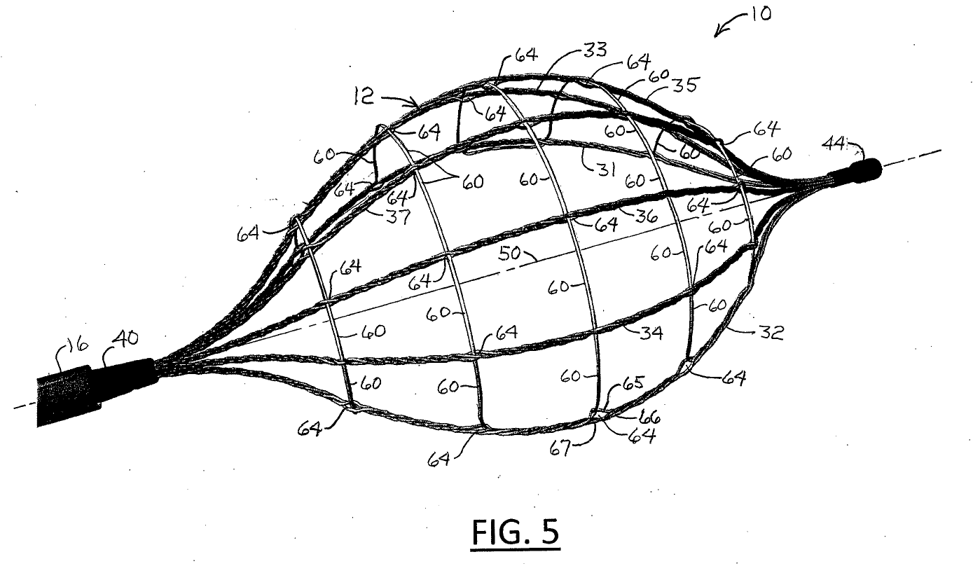

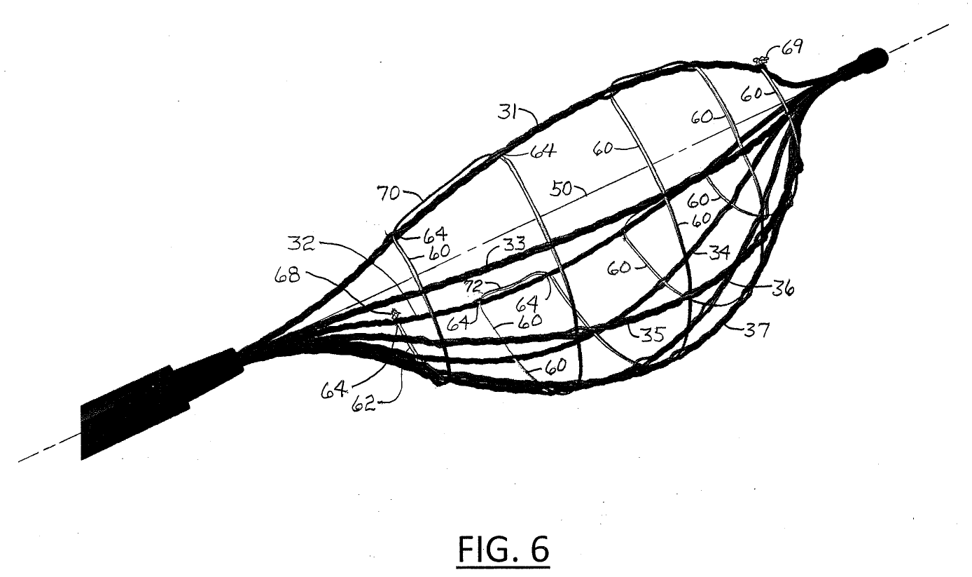

The flexible thread segments 60 can be attached to the wire frame members 31, 32, 33, 34, 35, 36, and 37 in myriad ways, one example of which is illustrated in FIGS. 1-6. In this example implementation, a single strand of flexible thread 62 is strung through a plurality of holes or eyes 64 in each of the wire frame members 31, 32, 33, 34, 35, 36, and 37, as perhaps best seen in FIGS. 5 and 6. In this example device 10, each wire frame member 31, 32, 33, 34, 35, 36, and 37 comprises a plurality of wire strands, e.g., strands 65, 66, 67, twisted together to provide an optimum semi-rigidity, shape memory, and resilience. The holes or eyes 64 are formed in the twisted wire frame members 31, 32, 33, 34, 35, 36, and 37 by spreading one of the strands 65, 66, 67 slightly away from the others, as best seen in FIG. 5. The single strand of flexible thread 62 can be strung or laced through the myriad holes in any manner or orientation to create spaces between thread segments 60 in any shape, but, in this example shown in FIGS. 1-6, the thread 62 is strung through the eyes 64 in a manner that creates rectangular spaces between the thread segments. It can be seen in several views, but perhaps best in FIG. 6, that a first end of the single strand of thread is provided with a knot 68 that anchors and prevents that end from sliding through the adjacent eye 64 in one of the top wire frame members, for example., the wire frame member 32. The thread 62 then extends transversely across the angular spaces between adjacent wire frame members 34, 36, 37, 35, and 33 and through eyes 64 in those wire frame members to the opposite top wire frame member 31, where it extends through an eye 64 and then parallel to that top wire frame member 31 to the next eye 64 on that wire frame member 31, as indicated at 70. From its extension through that next eye 64 in the wire frame member 31, the thread 62 then extends transversely back across the spaces between, and through respective eyes 64 in, the adjacent wire frame members 33, 35, 37, 36, and 34 to another eye 64 in the top wire frame member 31, where it extends through that eye 64 and then parallel to that top wire frame member 32, as indicated at 72. That pattern continues back and forth from one top wire frame member to the other via the intermediate wire frame members to a last eye 64 near the distal end of the basket 12, where the thread terminates in another knot 69 that anchors that terminated end to the wire frame member 31. The opposite ends of the single thread 62 could be anchored to any one or two of the wire frame members 31, 32, 33, 34, 35, 36, and 37. It is advantageous for the length of the thread 62 between the knots 68, 69 to be just the amount that will cause the thread 62 and its segments 60 to become somewhat taught when the basket 12 is fully expanded for the reasons explained above.

The spacing of the holes or eyes 64 in the wire frame members 31, 32, 33, 34, 35, 36, and 37 set the sizes of the rectangular spaces between the thread segments 60. Persons skilled in the art can provide such spacings to be optimally effective for whatever kinds of endoscopic procedures or applications the retrieval device 10 is to be used. In general, it is desirable to provide the spacings at optimal distances to capture and hold the target tissue pieces or other materials while allowing liquids and other smaller materials to flow through spaces between the thread segments 64 and adjacent wire frame members 31, 32, 33, 34, 35, 36, and 37.

Other methods and instrumentalities can also be used for fastening the flexible thread segments 60 onto the wire frame members 31, 32, 33, 34, 35, 36, and 37. For example, the thread can be tied, adhered with surgical glue, or otherwise attached to wire frame members 31, 32, 33, 34, 35, 36, and 37. Also, small inserts (not shown) with holes or eyes can be wound into and between the wire strands 65, 66, 67 of the wire frame members 31, 32, 33, 34, 35, 36, and 37. Also, the wire frame members 31, 32, 33, 34, 35, 36, and 37 can be single strand wires or cords with eyes molded or formed into them. The thread 62 or thread segments 60 can be cotton, nylon, polyester, or any other myriad other flexible thread materials. In some embodiments, the top wire frame members 31, 32 can be heavier or stronger wires, strands, or cords than the other wire frame members 33, 34, 35, 36, 37 in order enhance the ability of those top wire frame members 31, 32 to hold the open shape of the mouth as the basket 12 is rotated and manipulated during endoscopic procedures. For example, but not for limitation, the strands 65, 66, 67 of the wire frame members 31, 32, 33, 34, 35, 36, and 37 can be in a range of 0.02 inch to 0.20 inch. In one embodiment the strands 65, 66, 67 for the top wire frame members 31, 32 are 0.15 inch, and the strands 65, 66, 67 for the other wire frame members 33, 34, 35, 36, and 37 are 0.12 inch.

The foregoing description provides examples that illustrate the principles of the invention, which is defined by the features that follow. Since numerous insignificant modifications and changes will readily occur to those skilled in the art once they understand the invention, it is not desired to limit the invention to the exact example constructions and processes shown and described above. Accordingly, resort may be made to all suitable combinations, subcombinations, modifications, and equivalents that fall within the scope of the invention as defined by the features. The words “comprise,” “comprises,” “comprising,” “include,” “including,” and “includes” when used in this specification, including the features, are intended to specify the presence of stated features, integers, components, or steps, but they do not preclude the presence or addition of one or more other features, integers, components, steps, or groups thereof The terms upper, upwardly, lower, bottom, top, down, downwardly, vertical, horizontal, and other directional terms in this description are in reference to the diagrammatic orientations depicted in the drawings and are only used for convenience and clarity in this description unless otherwise indicated. They are not intended to limit the trap 10 to any particular orientation in real use applications, and, in fact, the trap 10 can be positioned and used in any desired orientation. Also, while the description was directed to trapping nickel from nickel-containing precursor gas for convenience, the trap 10 can also be used for trapping and removing other materials from effluent gas flow streams.

1. Surgical retrieval apparatus, comprising:
a plurality of bowed, resiliently deformable, wire frame members with bow shaped memory fastened together at their proximal ends in immovable relation to each other and to a tether and extending in radially flared formation at angularly spaced positions in relation to each other; and
a plurality of flexible thread segments extending transversely across the angular spaces between adjacent ones of the wire frame members and spaced longitudinally from each other.

2. The retrieval apparatus of claim 1, wherein two of the wire frame members flare radially outward in diametrically opposite directions from each other and a plurality of other wire frame members bow radially outward at angular spacings of less than 180 degrees to form a basket with a 180 degrees wide open mouth.

3. The retrieval apparatus of claim 2, wherein the wire frame members have eyes spaced longitudinally on the wire frame members, and a single thread comprises the thread segments, wherein the single thread is laced through the eyes and transversely across the angular spaces between adjacent ones of the wire frame members.

4. The retrieval apparatus of claim 3, wherein the single thread is anchored at each of its ends to one or more of the wire frame members with just enough length between the anchored ends so that the single thread becomes taut when the wire frame members are fully extended in their bowed shapes.

5. The retrieval apparatus of claim 3, wherein the wire frame members comprise multiple wire strands wound together, and wherein the eyes are formed by spreading one of the strands in relation to the other strands in a wire frame member.

6. The retrieval apparatus of claim 2, wherein the flexible thread segments are attached to the wire frame members with surgical glue.

7. The retrieval apparatus of claim 2, wherein the flexible thread segments are attached to the wire frame members with knots.

8. A method of forming an endoscopic retrieval device, comprising:
fastening a plurality of bowed, semi-rigid, resiliently collapsible wire frame members together in immovable relation to each other at both their proximal and distal ends such that the bowed wire frame members flare radially outward in relations to each other in angularly spaced relation to each other;
connecting the proximal ends of the wire frame members to a distal end of a tether; and
extending a plurality of flexible thread segments transversely across the angular spaces between adjacent ones of the wire frame members.

9. The method of claim 8, including providing a plurality of eyes at spaced distances longitudinally on the wire frame member, and lacing a thread comprising the thread segments through the eyes.

10. The method of claim 9, wherein the wire frame members comprise a plurality of wound wire strands, and including providing the eyes in the wire frame members by spreading one of the wire strands in relation to one or more of the other wire strands at longitudinally spaced distances along the wire frame members.

11. The method of claim 8, including providing the thread segments in lengths that become taut when the wire frame members are deployed in fully flared relation to each other.

12. The method of claim 9, including lacing a thread comprising the thread segments through the eyes to extend between adjacent ones of the wire frame members at multiple longitudinal locations traversing the angular spacings between the wire frame members.

13. The method of claim 8, including anchoring the flexible thread segments to the wire frame members with medical glue.
